(19) United States
(12) Patent Application Publication
(10) Pub. No.: US 2006/0210994 A1
(43) Pub. Date: Sep. 21, 2006

(54) Microfluidic systems and methods for using microfluidic devices

(76) Inventor: Timothy Herbert Joyce, Mountain View, CA (US)

Correspondence Address: AGILENT TECHNOLOGIES, INC.;Intellectual Property Administration, Legal Department, DL429, P. O. Box 7599, Loveland, CO, 80537-0599, US

(21) Appl. No.: 11/080,181

(22) Filed: Mar. 15, 2005

(51) Int. Cl. C12Q 1/68; G06F 19/00

(52) U.S. Cl. 435/6; 204/450; 702/20; 977/924

(57) ABSTRACT

Microfluidic systems and methods of their use are provided.

### BACKGROUND

Microfluidic devices are becoming useful in a wide variety of applications apart from their historic uses in ink-jet printers and lab-on-a-chip assays. Potential applications include pharmaceuticals, biotechnology, the life sciences, defense, public health, and agriculture. In general, microfluidics refers to a set of technologies that control the flow of minute amounts of liquids or gases (collectively referred to as fluids) in a miniaturized system. A microfluidic device usually contains one or more channels with at least one dimension less than 1 mm. Common fluids used in microfluidic devices include whole blood samples, bacterial cell suspensions, protein or antibody solutions and various buffers. Microfluidic devices can be used to obtain a variety of measurements including molecular diffusion coefficients, fluid viscosity, pH, chemical binding coefficients, and enzyme reaction kinetics. Other applications for microfluidic devices include capillary electrophoresis, isoelectric focusing, immunoassays, flow cytometry, sample injection of proteins for analysis via mass spectrometry, PCR amplification, DNA analysis, cell manipulation, cell separation, cell patterning, and chemical gradient formation. Many of these applications are useful in clinical diagnostics. A particular application of microfluidic devices is in biopolymer analysis, for example sequencing of polynucleotides or the detection of a polynucleotide, polypeptide, or other biopolymer.

Processing of samples for analysis by detection devices often includes pretreatment of the sample to condition the sample for analysis. For example, electrophoresis is typically performed on a heterogeneous sample to fractionate the sample into discrete fractions. Unfortunately, electrophoresis of a sample may be problematic for certain detection devices, for example detection devices monitoring conductivity changes. Electrophoretic buffers can interfere with conductivity measurements due in part to the ionic content of the electrophoretic buffers. One example of a detection device that monitors conductivity includes resonant tunneling electrodes.

Resonant tunneling electrodes measure changes in conductivity when current carriers in the electrodes are relatively similar to the energy levels of the proximal analyte, for example a polynucleotide. Tunneling refers to the movement of an electron from a first position in space to a second position in space through a region that would be energetically excluded without quantum mechanical tunneling. For resonant tunneling to be effective, the conductivity of the solution containing the analyte should not interfere with measuring the tunneling current.

Resonant tunneling has been described in the sequencing of biopolymers in US Patent Application Publication No. 20040144658 to Flory, which is incorporated herein by reference in its entirety. Flory teaches that variations in the position of the analyte with regard to a set of resonant tunneling electrodes can cause changes in the magnitude of the tunneling current which would be far in excess of the innate differences expected between different base-types under ideal conditions. Thus, sequencing with resonant tunneling electrodes can result in sequencing errors due to fluctuations in the conductivity of the analyte solution.

Accordingly, there is a need for systems and methods that improve the accuracy and reliability of resonant tunneling current detection, for example improved resonant tunneling analysis systems and methods.

### SUMMARY

Microfluidic systems and methods of their use are provided. An exemplary microfluidic system, among others, includes a sample preparation device and a microfluidic in fluid communication with the sample preparation device. The microfluidic device includes at least one set of resonant tunneling electrodes disposed in a microchannel. The resonant tunneling electrodes configured to detect an analyte.

Another exemplary microfluidic system, among others, includes a capillary electrophoretic device and a resonant tunneling electrode. The microchannel is operably coupled to a microchannel of a microfluidic device and is configured to receive electrophoretically separated analytes. The resonant tunneling electrode disposed in the microchannel for detecting at least one of the separated analytes.

An method for sequencing a target polynucleotide, among others, includes: electrophoretically separating a plurality of polynucleotides based on at least one characteristic of the polynucleotides; receiving the separated polynucleotides into a microfluidic channel; and determining a statistically significant sequence of the target polynucleotide by detecting tunneling current through the each of the polynucleotides with a resonant tunneling electrode and correlating the detected current to predetermined currents indicative of specific polynucleotides.

### BRIEF DESCRIPTION OF THE DRAWINGS

Reference is now made to the following drawings. Note that the components in the drawings are not necessarily to scale.

FIG. 1 shows a schematic of an exemplary embodiment of a microfluidic system.

FIG. 2 shows a diagram of an exemplary embodiment of a microfluidic device.

FIG. 3 shows a diagram of an alternative embodiment of a microfluidic device.

FIG. 4A shows a diagram of an exemplary sample preparation device.

FIG. 4B shows an alternative view of a capillary serving as a sample preparation device.

FIG. 5 shows a diagram of another embodiment of a microfluidic system.

FIG. 6 shows a diagram of an alternative embodiment of a microfluidic system.

FIG. 7 is a flow diagram of an exemplary method for characterizing an analyte according to the present disclosure.

### DETAILED DESCRIPTION

Definitions

The term “nanopore” refers to an opening of 100 nm or less at its widest point. The aperture can be of any geometric shape or configuration, including, but not limited to, square, oval, circular, diamond, rectangular, star, or the like.

The term “polymer” refers to a composition having two or more units or monomers attached bonded, or physically associated to each other. The term polymer includes biopolymers.

A “biopolymer” refers to a polymer of one or more types of repeating units. Biopolymers are typically found in biological systems and particularly include polysaccharides (such as carbohydrates), peptides (which term is used to include polypeptides and proteins), glycans, proteoglycans, lipids, sphingolipids, known biologicals materials such as antibodies, etc. and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in hydrogen bonding interactions, such as Watson-Crick type, Wobble type and the like. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

“Electrophoresis” refers to the motion of a charged particle or polymer, for example colloidal particle, under the influence of an electric field.

“Entangled polymer solutions” refers to solutions in which polymers will interpenetrate each other. This causes entanglements and restricts the motion (reptation) of the molecules to movement along a ‘virtual tube’ that surrounds each molecule and is defined by the entanglements with its neighbors.

The term “gel” refers to a network of either entangled or cross-linked polymers swollen by solvent. The term is also used to describe an aggregated system of colloidal particles that forms a continuous network.

“Statistically significant” refers to a result if it is unlikely to have occurred randomly. “Significant” means probably true (not due to chance). Generally, a statistically significant sequence means a sequence that has about a 5% or less probability of including a random sequence error.

A “microfluidic device” refers to a device that has one or more channels with at least one dimension less than 1 mm. Common fluids used in microfluidic devices include, but are not limited to, whole blood samples, serum, plasma, cellular extracts, bacterial cell suspensions, protein solution, antibody solutions and/or various buffers.

As will be described in greater detail here, embodiments of microfluidic systems and methods of use thereof are provided.

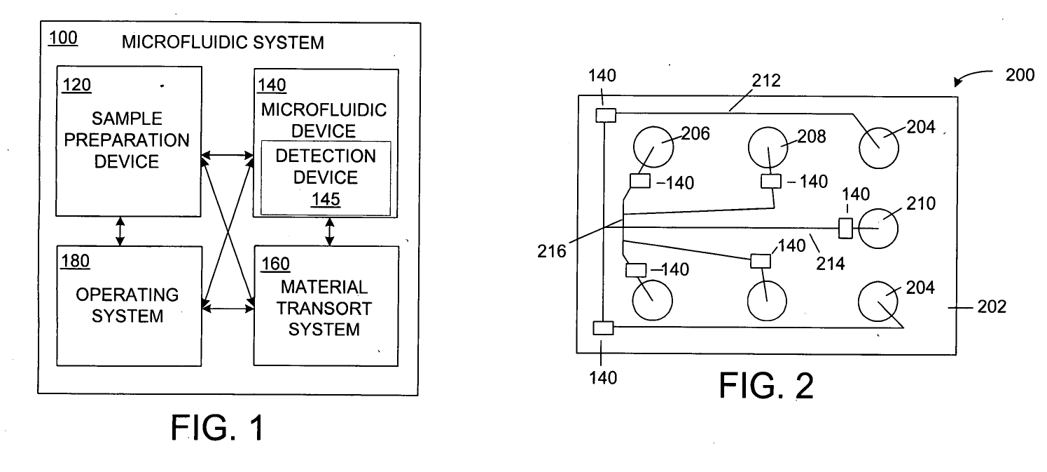

FIG. 1 shows a graphical representation of an exemplary microfluidic system 100 of the present disclosure. The microfluidic system 100 can include a sample preparation device 120, a microfluidic device 140, a material transport system 160, and an operating system 180, each of which can be in communication with and/or operably linked to each other. For example, the sample preparation device 120 is in fluid and optionally, electrical communication with the microfluidic device 140, for example a microchannel. The microchannel is in fluid communication with a detection device 145. Characteristics of the sample can be detected, monitored, and collected using the detection device 145. The detection device 145 includes, but is not limited to, a resonant tunneling electrode.

The microfluidic device 140 also comprises the material transport system 160. The material transport system 160 includes, but is not limited to, electrokinetic components, electroosmotic components, electrophoretic, and/or other fluid manipulation components (e.g., micro-pumps and microvalves, fluid switches, fluid gates, etc.) sufficient for the movement of material within the microfluidic device 140.

The microfluidic system 100 optionally includes an operating system 180 that can be operatively linked to the sample preparation device 120, the microfludic device 140, and/or the material transport system 160. The operating system 180 includes, but is not limited to, electronic equipment capable of measuring characteristics of an analyte such as a polymer, for example a polynucleotide, as the analyte travels along a channel, a computer system capable of controlling the measurement of the characteristics and storing the corresponding data, control equipment capable of controlling the conditions of the detection device, and components that are included in the detection device 145 that are used to perform the measurements as described below. The microfluidic system 100 can also be in communication with a distributed computing network such as a LAN, WAN, the World Wide Web, Internet, and/or intranet.

The detection device 145 can measure characteristics such as, but not limited to, the amplitude or duration of individual conductance or electron tunneling current changes as an analyte, such as a polymer, passes near or through the detection device 145. US Patent Application Publication Nos. 20040149580 and 20040144658 to Flory disclose the use of resonant tunneling electrodes for the characterization of biopolymers and are incorporated by referenced in their entireties.

In one embodiment of the present disclosure, an analyte can be characterized by measuring quantum mechanical tunneling currents through the portion of the analyte as it passes between a pair of electrodes. Tunneling current has an exponential dependence upon the height and width of the quantum mechanical potential barrier to the tunneling process. This dependence implies an extreme sensitivity to the precise location of the analyte with respect to the set of tunneling electrodes. Both steric attributes and physical proximity to the tunneling electrode could cause changes in magnitude of the tunneling current which making more difficult the job of accurately characterizing the analyte, for example sequencing a polynucleotide.

Typically, conductance occurring through an analyte as it traverses the detection device 145 is detected or quantified. More specifically, electron tunneling conductance measurements are detected for each monomer of a polymeric analyte as each monomer traverses the detection device 145. Such measurements include, but are not limited to, changes in data, which can identify the monomers in sequence, as each monomer can have a characteristic conductance change signature. For instance, the volume, shape, purine or pyrimidine base, or charges on each monomer can affect conductance in a characteristic way. Likewise, the size of the entire polynucleotide can be determined by observing the length of time (duration) that monomer-dependent conductance changes occur. Alternatively, the number of nucleotides in a polynucleotide (also a measure of size) can be determined as a function of the number of nucleotide-dependent conductance changes for a given nucleic acid traversing the detection device. The number of nucleotides may not correspond exactly to the number of conductance changes, because there may be more than one conductance level change as each nucleotide of the nucleic acid passes sequentially through the detection device 145. However, there can be proportional relationship between the two values which can be determined by preparing a standard with a polynucleotide having a known sequence.

FIG. 2 shows a diagram of a representative microfluidic device 200. The microfluidic device 200 is an alternative embodiment of the microfluidic device 140 shown in FIG. 1. In this embodiment, a housing 202 includes a plurality of wells or sample inlets 204. The sample inlet 204 is generally configured to receive electrophoretically separated samples from the sample preparation device 120 (e.g., an electrophoretic device). The sample inlet 204 is connected to a well 210 by the channels 212 and 214. The well 210 can serve as a receptacle that receives analyzed or processed samples. In operation, a sample is received into the sample inlet 204 from a sample preparation device such as a capillary electrophoresis device. Alternatively, the inlet 204 can function as a sample preparation device by modulating the sample to optimize the sample for detection. In one embodiment, the inlet 204 modulates the ionic content of the sample so that ions in the sample buffer do not interfere with characterization of analytes in the sample by resonant tunneling electrodes. The material transport system 216 moves the sample through the channel 212 and throughout the microfluidic device. The channel 212 can also comprise a separation matrix for assisting in the further sorting of the sample.

The resonant tunneling electrode 145 can be configured to monitor conductance, for example tunneling current, of analytes as the analytes pass though the resonant tunneling electrode. Generally, at least one resonant tunneling electrode is operably coupled to the microfluidic device 200.

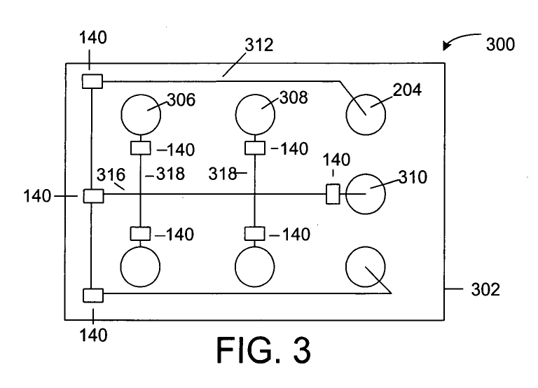

FIG. 3 shows a diagram of an alternative embodiment of the microfluidic device 300. In this embodiment, a plurality of channels intersect at “T” junctions as well as optionally containing at least one turn or bend. Segments of a sample can be drawn into different channels for different analysis as the sample passes each T junction. Alternatively, different reagents can be delivered to the microfluidic device through inlets 306 or 308. For example, the inlets 306 or 308 can provide buffering agents containing ion chelating agents to reduce the concentration of ions in the sample.

Having generally described an exemplary microfluidic system, the components of a representative microfluidic system will be described in more detail.

Sample Preparation Device

In one embodiment, the sample preparation device 120 advantageously sorts and optionally groups or stacks similar analytes, for example analytes of a specific mass or range of masses, molecular weight, size, charge, conformation including single or double stranded conformations, or charge-to-mass ratio to be detected, by a detection device 145, for example a resonant tunneling electrode. Providing multiple analytes of similar or identical characteristics allows for collection of multiple data points for the same analyte. The multiple data points can be analyzed to increase the fidelity of the result. Some data points may incorrectly represent a characteristic of the analyte being analyzed. Incorrect, or outlying data points can be ignored or deleted from the data set to produce a more reliable and statistically significant result.

In another embodiment, the sample preparation device 120 receives separated analytes or analytes that have been processed. Generally, the processed analytes are in a buffer solution. The sample preparation device 120 can modify the buffer solution containing the analytes to prepare the analytes for detection by the detection device 145. In one embodiment, the sample preparation device 120 modulates the ionic concentration, pH, temperature, viscosity, concentration, or a combination thereof to optimize conditions for detection of the analytes by the detection device 145. For example, the sample preparation device 120 can be a microchamber disposed in the microfluidic device. The microchamber can serve as a desalting chamber to remove excess ions or other components that may interfere with detection or characterization of the analytes.

Sample Sorting and Stacking

In some embodiments, a plurality of analytes may be sorted, stacked, or separated with the sample preparation device 120 using conventional techniques including, but not limited to, electrophoresis, capillary electrophoresis, molecular sieves, antibody capture, chromatography, affinity chromatography, polynucleotide capture, chromatography, reverse phase chromatography, and ion exchange chromatography.

In one embodiment, capillary electrophoresis can also be performed in one more microfluidic channels. A microfluidic channel includes, but is not limited to, a surface micro-machined labyrinth having one central inlet and at least one outlet. These separations are facilitated by the use of high voltages, which may generate electroosmotic flow, electrophoretic flow, or a combination thereof, of buffer solutions and ionic species, respectively, within the channel. The properties of the separation and the ensuing electropherogram have characteristics resembling a cross between traditional polyacrylamide gel electrophoresis (PAGE) and modern high performance liquid chromatography (HPLC). In one embodiment, the sample preparation deivce 120 utilizes a high electric field strength, for example, about 500 V/cm or more. One process that drives CE is electroosmosis. Electroosmosis is a consequence of the surface charge on the wall of the channel. Some channels that are typically used for separations have ionizable silanol groups in contact with the buffer contained within the channel. The degree of ionization can be controlled mainly by the pH of the buffer.

A negatively-charged channel wall attracts positively-charged ions from the buffer, creating an electrical double layer. When a voltage is applied across the channel, cations in the diffuse portion of the double layer migrate in the direction of the cathode, carrying water with them. The result is a net flow of buffer solution in the direction of the negative electrode. In untreated microchannels most solutes migrate towards the negative electrode regardless of charge when the buffer pH is above 7.0.

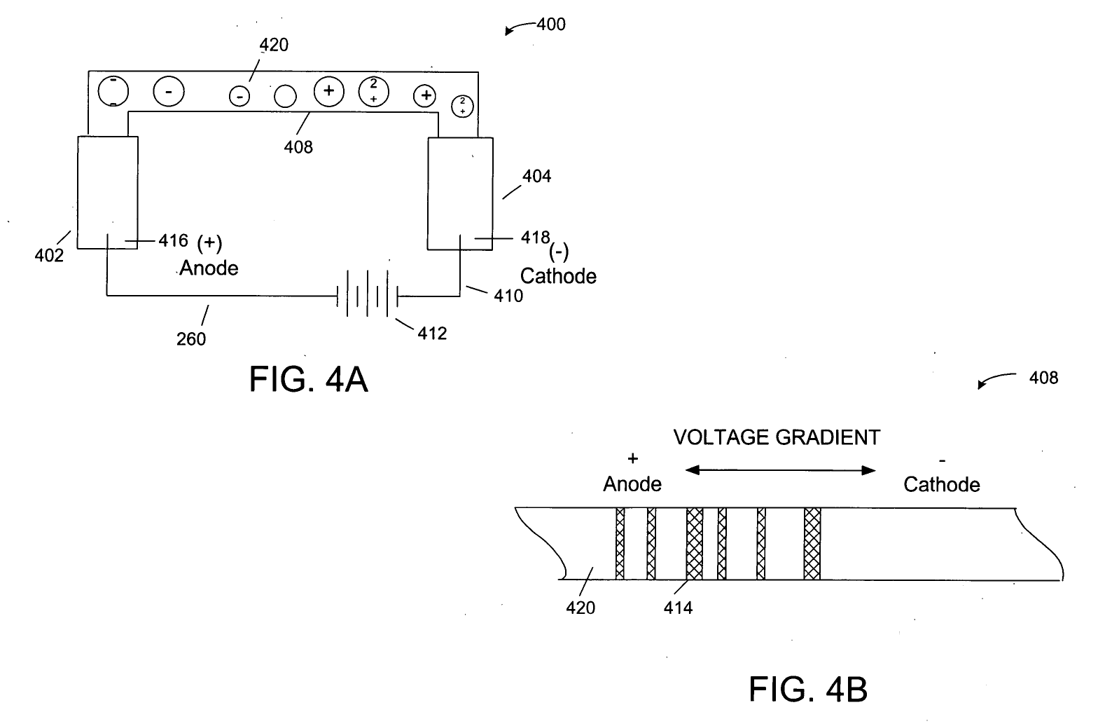

Capillary electrophoresis includes, but is not limited to, capillary zone electrophoresis, isoelectric focusing, capillary gel electrophoresis, isotachophoresis, and micellar electrokinetic capillary chromatography. Capillary zone electrophoresis (CZE), also known as free solution capillary electrophoresis, is the simplest form of CE. The separation mechanism is based on differences in the charge-to-mass ratio. Fundamental to CZE are homogeneity of the buffer solution and constant field strength throughout the length of the capillary. Following injection and application of voltage, the components of a sample mixture separate into discrete zones 414 as shown in FIG. 4B.

With isoelectric focusing (IEF) a molecule will migrate so long as it is charged, and will stop when it becomes neutral. IEF is run in a pH gradient where the pH is low at the anode and high at the cathode. The pH gradient is generated with a series of zwitterionic chemicals known as carrier ampholytes. When a voltage is applied, the ampholyte mixture separates in the capillary. Ampholytes that are positively charged will migrate towards the cathode while those negatively charged migrate towards the anode. It will be appreciated that the pH of the anodic buffer must be lower than the isoelectric point of the most acidic ampholyte to prevent migration into the analyte. Likewise, the catholyte must have a higher pH than the most basic ampholyte.

Nucleic acids are generally electrophoresed in neutral or basic buffers—as anions with their negatively charged phosphate groups. For small DNA fragments, e.g., nucleosides, nucleotides, and small oligonucleotides, free-solution techniques (CZE, MECC) can be applied—generally in conjunction with uncoated capillaries or microfluidic channels. Alternatively, separation of larger deoxyoligonucleotides can be accomplished using capillary gel electrophoresis, generally with coated capillaries or microfluidic channels, in which, as the name implies, the capillary or channel is filled with an anticonvective medium such as polyacrylamide or agarose. The gel suppresses electroosmotic flow and acts a sieve to sort analytes by size. Oligonucleotides, for example poly(dA)40-60 can be separated using this method with a gel of 8% monomer and a buffer including 100 mM Tris-borate, pH 8.3 with 2 mM EDTA and 7 M urea, in under 35 min with unit base resolution.

Isotachophoresis relies on zero electroosmotic flow, and the buffer system is heterogeneous. This is a free solution technique, and the capillary or channel is filled with a leading electrolyte that has a higher mobility than any of the sample components to be determined. Then the sample is injected. A terminating electrolyte occupies the opposite reservoir, and the ionic mobility of that electrolyte is lower than any of the sample components. Separation will occur in the gap between the leading and terminating electrolytes based on the individual mobilities of the analytes.

Micellar electrokinetic capillary chromatography (MECC) is a free solution technique that uses micelle-forming surfactant solutions and can give rise to separations that resemble reverse-phase liquid chromatography with the benefits of capillary electrophoresis. Unlike isoelectric focusing, or isotachophoresis, MECC relies on a robust and controllable electroosmotic flow. MECC takes advantage of the differential partitioning of analytes into a pseudo-stationary phase including of micelles. Ionic, nonionic and zwitterionic surfactants can be used to generate micelles. Representative surfactants include, but are not limited to, SDS, CTAB, Brij, and sulfobetaine. Micelles have the ability to organize analytes at the molecular level based on hydrophobic and electrostatic interactions. Even neutral molecules can bind to micelles since the hydrophobic core has very strong solubilizing power.

FIG. 4A shows an exemplary electrophoretic device 400 using a voltage gradient or an electric field to separate various analytes. A sample inlet 402 receives a sample, for example a sample containing a plurality of polynucleotides. The sample is preferably a fluid sample in a solution buffered to a desired pH and ionic strength. Generally, the electrophoretic device has an anode buffer in the inlet 402 and a cathode buffer in an outlet 404. It will be appreciated that the pH of the buffer and ionic strength can each be modulated, which in turn can modulate the electrophoretic separation of analytes. Additionally, viscosity builders, surfactants, denaturing agents, or other additives can be added to the sample or buffer to vary the separation resolution of the analytes. In some embodiments, capillary electrophoresis require modifications to the walls of the capillaries or channels, for example channels of fused silica. The walls can be modified, for example, to modify or suppress electroosmotic flow, and/or to reduce unfavorable wall-analyte interactions. In one embodiment, the electrophoretic device 400 does not exert electroosmotic flow on the polymers to be separate. For example, a tube or the microchannel 408 can have surfaces that are neutral or uncharged during electrophoresis. Charged surfaces of the tube or the microchannel 408 can optionally be coated, for example with an ionic surfactant such as a cationic or anionic surfactant. Exemplary coating substances or buffer additives include, but are not limited to, SDS, cetyltrimethylammonium bromide (CTAB), polyoxyethylene-23-lauryl ether; sulfobetaine (BRIJ), TWEEN, MES, Tris, CHAPS, CHAPSO, methyl cellulose, polyacrylamide, PEG, PVA, methanol, acetonitrile, cyclodextrins, crown ethers, bile salts, urea, borate, diaminopropane, and combinations thereof. Neutralizing charged surfaces of the channel 408 can eliminate or reduce electroosmotic flow. Alternatively, modifications to the surface of the channel 408 or to the buffers can eliminate or reverse the electroosomotic force. For example, neutral deactivation with polyacrylamide eliminates the electroosmotic. This results from a decreased effective wall charge and increased viscosity at the wall. Deactivation with cationic groups can reverse the electroosomotic flow, and deactivation with amphoteric molecules allows one to control the direction of the electroosmotic force by altering the pH.

A wide variety of covalent and adsorbed capillary or microfluidic channel coatings that completely suppress electroosmotic flow are known in the art and are commercially available. Coatings that include covalently bound or adsorbed neutral polymers, such as linear polyacrylamide, are highly stable, resist a variety of analytes, and reduce electroosmotic flow to almost undetectable levels. Such coatings are useful for a wide range of applications including DNA and protein separations, with the requirement that all analytes of interest migrate in the same direction (e.g., have charge of the same sign). For many other applications, however, electroosmotic flow can be used as a pump to mobilize analytes of both positive and negative charge (e.g., a mixture of proteins with a wide range of isoelectric points), or to mobilize species with very low electrophoretic mobility. Bare fused silica capillaries exhibit strong cathodal electroosmotic flow, but are prone to adsorption of analytes, leading to irreproducible migration times and poor peak shapes.

In one embodiment, electrophoretic separation of analytes occurs in the tube, the capillary, or the microfluidic channel 408. The tube or the microfluidic channel 408 can be coated or uncoated. In another embodiment, exemplary microfluidic channels have a diameter of less than 1 mm, typically less than about 500 μm, more typically less than about 200 μm or from about 1 μm to about 100 μm. FIG. 4B is sectional view of the tube 408 showing the bands or the zones 414 of analytes as they are separated along a voltage gradient. Generally, the samples move from anode to cathode, however, it will be appreciated that the polarity can be reversed by changing the buffer system, adding ionic surfactants to the sample or buffer or coating the interior surfaces of the capillary to reduce or eliminate electroosmotic flow. Typically, the analytes in one band 414 are of uniform size, uniform number of monomers, and optionally, uniform sequence.

The power source 412 (FIG. 4A) provides a voltage gradient between the anode 416 and the cathode 418. The power source 412 is in electrical communication with the wells 402, 404 using convention electrical conductors 410. Generally, the power source 412 can supply about 10 to about 60 kV, typically about 30 kV. It will be appreciated that the voltage can be adjusted to modify analyte separation. When a voltage gradient is established, individual analytes will move along the voltage gradient, for example according to their charge, mass, or charge-to-mass ratio. In conventional capillary electrophoresis, small positively charged analytes will move quickly from the anode towards the cathode. Larger positively charged analytes follow with large negatively charged analytes traveling at the end of the sample.

During separation, the analytes travel through the channel 408. The channel 408 can be filled with a separation matrix 420 and a buffer to maintain ionic and pH conditions. The ionic and pH conditions can be optimized to increase the separation resolution of specific analytes. The different analytes may require different buffers, ionic concentrations, voltages, and separations times to achieve separation of a specific analyte or group of analytes. Representative separation matrices include, but are not limited to, polymers including polyacrylamides, methacrylates, polysiloxanes, agarose, agar, polyethylene glycol, cellulose, or any other substance capable of forming a meshlike framework or sieve. The separation matrix can be colloids, “polymer solutions,” “polymer networks,” “entangled polymer solutions,” “chemical gels,” “physical gels,” and “liquid gels.” More particularly, the separation matrix can be a relatively high-viscosity, crosslinked gel that is chemically anchored to the channel wall (“chemical” gel), or a relatively low-viscosity, polymer solution (“physical” gel). The mesh or sieve will work to retain or hinder the movement of large analytes; whereas, small analytes will travel quicker through the mesh. The analytes having similar or identical characteristics such as lengths, molecular weights, or charges, will stack together or travel in the bands 414. It will be appreciated that the size of the pores of the separation matrix can be varied to separate different analytes or groups of analytes.

In another embodiment, the inlet 402 or the channel 408 of the microfluidic device can be coated with a substance that specifically binds to a specific analyte or group of analytes. For example, a surface of the inlet 402 or the channel 408 can be coated with an antibody that specifically binds directly or indirectly to a specific analyte such as a polypeptide or polynucleotide. Alternatively, a polynucleotide having a predetermined sequence can be attached to a surface of the inlet 402 of the disclosed microfluidic device for binding complementary sequences present in a sample. Suitable polynucleotides are at least about 6 nucleotides, typically about 10 to about 20 nucleotides, even more typically, about 6 to about 15 nucleotides. It will be appreciated that any number of nucleotides can be used so long as the polynucleotide can specifically hybridize with its complementary sequence or polymers containing its complementary sequence.

Another embodiment provides a microfluidic device having polypeptides attached to an interior surface of a reservoir, tube, or channel. The attached polypeptides can specifically bind to another polypeptide. Exemplary attached polypeptides include; but are not limited to, polyclonal or monoclonal antibodies, fragments of antibodies, polypeptides, for example polypeptides that form dimers with other polypeptides, or polypeptides that specifically associate with other polypeptides or small molecules to form macromolecular complexes or complexes of more than one subunit.

In other embodiments, binding agents are attached to a matrix or resin that is placed inside a reservoir, tube, or channel. The binding matrix or resin can be replaced or recharged as needed. The resins or underlying matrices are generally inert and biologically inactive apart from the binding agent coupled thereto, and can be plastic, metal, polymeric, or other substrate capable of having a binding agent attached thereto. The binding agent can be a polypeptide, small organic molecule, nucleic acid, biotin, streptavidin, carbohydrate, antibody, or ionic compound, fragments thereof, or combinations thereof.

In one embodiment, the inlet 402 receives a plurality of analytes containing a target analyte. Analytes in the sample that are not the target analyte are captured by a binding molecule attached to the surface of the inlet 402, a tube, or channel of the microfluidic device and are immobilized. The target analyte is mobile and is transported through the microfluidic device.

In another embodiment, the target analyte is specifically immobilized by a binding agent such as a polypeptide or polynucleotide. Other analytes are flushed through the microfluidic device. Once the other analytes are separated from the target analyte, the target analyte is released from the binding agent, for example by changing pH, ionic strength, temperature, or a combination thereof. Data from the target analyte can then be captured by the microfluidic device as the target analyte travels through the microfluidic device.

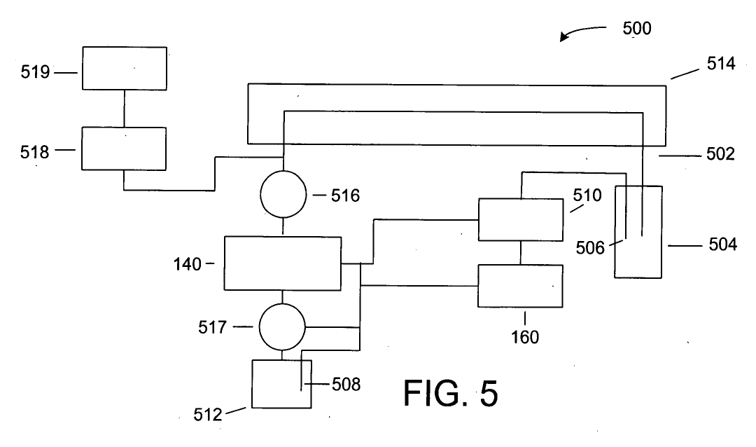

FIG. 5 is a diagram of an exemplary embodiment of a microfluidic system 500 comprising a temperature regulating device 514. In this embodiment, a microfluidic system 500 includes a capillary electrophoresis device 502 in fluid communication with the microfluidic device 140. It will be appreciated that the microfluidic system 500 can have one or more electrophoretic devices, each operatively coupled to one or more detection devices. The electrophoresis device 502 is typically at least one capillary, which can be coated, or non-coated, and optionally contains a separation matrix. A first end of the electrophoresis device 502 is immersed in the electrophoretic buffer of the reservoir 504. The cathode 506 of the power supply 510 is also immersed in the electrophoretic buffer of the reservoir 504. The anode 508 of the power supply 510 is typically immersed in a second electrophoresis buffer of a second reservoir 512. It will be appreciated that the anode 508 and the cathode 506 can be switched depending on the type of analyte to be separated and the electrophoretic conditions needed for separation. The power supply 510 establishes a voltage gradient between the cathode 506 and the anode 508.

The electrophoretic device 502 can optionally be housed or surrounded in whole or part by a temperature control device 514. The temperature control device 514 can be any refrigerating unit for dissipating or heating unit for generating heat. Such devices are known in the art and commercially available. In one embodiment, a temperature regulated fluid, for example a chilled fluid, is in contact with the electrophoretic device 502. The chilled fluid can be continuously recycled through a cooling device to control temperature during analyte separation.

In operation an analyte sample is delivered to the electrophoretic device 502. Representative delivery methods include, but are not limited to, pressure, electrical force, suction/vacuum, or gravity. Typically, an analyte sample is injected through an injection device, such as a syringe. The sample travels the length of the electrophoretic device 502 where the analyte sample is stacked into bands or plates, typically by size each analyte of the same size having the same sequence of monomers. Each analyte band or plate enters the microfluidic device 140 though the valve 516. The microfluidic device 140 is operably coupled to the resonant tunneling electrode 517. The resonant tunneling electrode 517 collects data from the analyte sample as each analyte traverse the detection device. Once the analyte is detected, it passes into a reservoir 512. The reservoir 512 can be any container comprising, but not limited to, 96 well plates and the like.

The icrofluidic analysis system 500 also optionally includes a replacement media 518 in fluid communication with the electrophoretic device 502 and a pump 519. If necessary, the pump 519 can pump the replacement media 518 into the electrophoretic device 502 to replace or recharge the electrophoretic device 502 separation matrix.

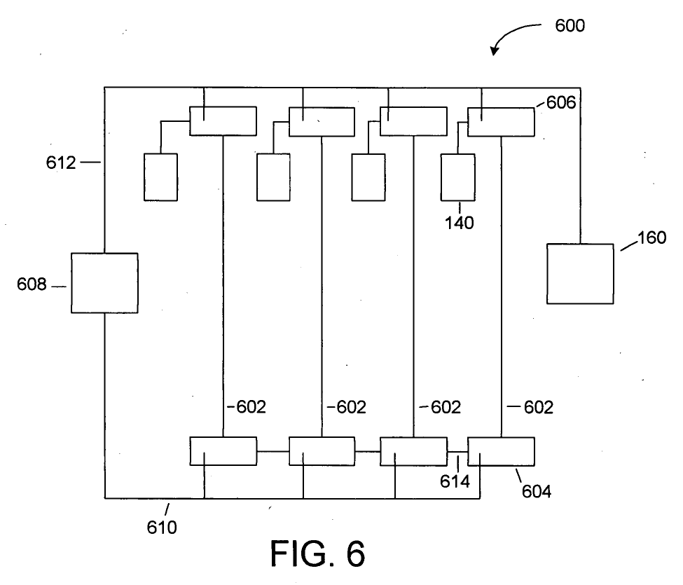

FIG. 6 shows an embodiment that provides a microfluidic system 600 having a plurality of electrophoretic devices 602, for example capillary tubes. A power source 608 establishes a voltage gradient between the reservoirs 604 and 606 to separate a analyte sample delivered to the electrophoretic device 602. The plurality of the reservoirs 604 can optionally be interconnected via a channel 614. The microfluidic device 140, for example a microchannel, receives stacked or separated analytes from the electrophoretic device 602. The microfluidic device includes a resonant tunneling electrode disposed in a microchannel. The resonant tunneling electrode collects data from each analyte as the analyte traverses the resonant tunneling electrode. The operating system 160 is optionally communicatively coupled to the microfluidic devices 140 and a power supply 608 and is configured to control operation of the system.

Reactions

Other embodiments provide reservoirs, wells, or modified tubes or channels, of the microfluidic device that are configured to perform, facilitate, or contain reactions, for example chemical or enzymatic reactions, on a sample containing a plurality of analytes. In one embodiment, a reservoir, channel or tube can be configured to perform polynucleotide amplification or primer extension using for example polymerase chain reaction (PCR). PCR and methods for performing PCR are known in the art. In order to perform PCR, at least a portion of the sequence of the DNA polymer to be replicated or amplified must be known. Short oligonucleotides (containing about two dozen nucleotides) primers that are precisely complementary to the known portion of the DNA polymer at the 3′ end are synthesized. The DNA polymer sample is heated to separate its strands and mixed with the primers. If the primers find their complementary sequences in the DNA, they bind to them. Synthesis begins (as always 5′−>3′) using the original strand as the template. The reaction mixture must contain all four deoxynucleotide triphosphates (dATP, dCTP, dGTP, dTTP) a DNA polymerase, for example a DNA polymerase that is not denatured by the high temperature needed to separate the DNA strands. Suitable heat stable DNA polymerases are known in the art and include, but are not limited to Taq polymerase. Polymerization continues until each newly-synthesized strand has proceeded far enough to contain the site recognized by a flanking primer. The process is repeated with each cycle doubling the number of DNA molecules. Using automated equipment, each cycle of replication can be completed in less than 5 minutes. After 30 cycles, what began as a single molecule of DNA has been amplified into more than a billion copies. It will be appreciated that Reverse Transcriptase PCR is also within the scope of this disclosure.

Other exemplary reactions include fragmenting analytes of a sample. Fragmenting a analyte can be accomplished enzymatically using proteases, peptidases, endonucleases, exonucleases, ribonucleases, physical shearing, sonication, and combinations thereof. Reagents for fragmenting analytes, for example nucleic acids and proteins are known in the art and are commercially available.

Detection Device

In one embodiment, the detection device 145 is operably coupled to the microfluidic device, for example a microfluidic channel, and can be configured to monitor, collect, transmit, or detect data concerning an analyte. A plurality of analytes of the same sequence can be delivered to detection device 145 in discrete amounts or bands from electrophoretic device 120, for example a microchannel. In still another embodiment, detection device 145 is electrically insulated from electrophoretic device 120, while optionally remaining in fluid communication with electrophoretic device 120. Electrically insulating the two components allows for different voltage gradients to be applied in the different components. In another embodiment, electrophoretic device 120 is in electrical communication with detection device 145 such that the voltage gradient maintained in electrophoretic device 120 is also maintained in detection device 145. In still another embodiment, the polarity of electrophoretic device 120 is maintained in detection device 145. In another embodiment, the polarity of electrophoretic device 120 is different than the polarity of detection device 145.

As noted, detection device 145 also includes a detector, such as an electrode or other sensing device, for collecting data from the analyte as it traverse a microchannel. The detectors can be configured to detect or collect different types of data as the analyte passes through a microchannel, including but not limited to, conductivity, ionic current, tunneling current, temperature, resistance, impedance, fluorescence, radioactivity, or a combination thereof. The data collected, recorded, or transmitted by the detectors can be correlated to specific monomers such that the sequence of monomers forming the analyte can be ascertained. For example, the data obtained from monomers of a specific analyte can be correlated to predetermined values indicative of a specific monomer. The predetermined values can be calculated or determined from analytes of a known sequence of monomers.

Detectors

As noted above, the microfluidic system 100 includes at least one detector that is configured to collect data as an analyte or polymer traverse a channel of a disclosed microfluidic device. The data can be used to determine the sequence of monomers forming the polymer. The data can be electromagnetic, conductive, colorometric, fluorometric, radioactive response, or a change in the velocity of electromagnetic, conductive, colorometric, fluorometric or radioactive component. Detectors can detect a labeled compound, with typical labels including fluorographic, colorometric and radioactive components. Exemplary detectors include analytical instruments such as NMR, mass spectrometer, IR detectors, resonant tunneling electrodes, spectrophotometers, photodiodes, microscopes, scintillation counters, cameras, film and the like, as well as combinations thereof. Examples of suitable detectors are widely available from a variety of commercial sources known to persons of skill.

In one embodiment, the detection system is an optical detection system and detects for example, fluorescence based signals. The detector may include a device that can expose a polymer with an exciting amount of electromagnetic radiation in an amount and duration sufficient to cause a fluorophore to emit electromagnetic radiation. Fluorescence is then detected using an appropriate detector element, e.g., a photomultiplier tube (PMT). Similarly, for screens employing colorometric signals, spectrophotometric detection systems are employed which detect a light source at the sample and provide a measurement of absorbance or transmissivity of the sample.

Other embodiments provide a detection system having non-optical detectors or sensors for detecting a particular characteristic or physical parameter of the system or polymer. Such sensors optionally include temperature (e.g., when a reaction produces or absorbs heat, or when the reaction involves cycles of heat as in PCR or LCR), conductivity, potentiometric (pH, ions), amperometric (for compounds that can be oxidized or reduced, e.g., O₂, H₂O₂, I₂, oxidizable/reducible organic compounds, and the like).

Still other detectors are capable of detecting a signal that reflects the interaction of a receptor with its ligand. For example, pH indicators which indicate pH effects of receptor-ligand binding can be incorporated into the device along with the biochemical system, i.e., in the form of encapsulated cells, whereby slight pH changes resulting from binding can be detected. Additionally, the detector can detect the activation of enzymes resulting from receptor ligand binding, e.g., activation of kinases, or detect conformational changes in such enzymes upon activation, e.g., through incorporation of a fluorophore which is activated or quenched by the conformational change to the enzyme upon activation. Such reporter molecules include, but are not limited to, molecular beacons.

Resonant Tunneling Electrode

Another embodiment provides a microfluidic system comprising a resonant tunneling electrode. Resonant tunneling electrodes and methods of their use in characterizing biopolymers are disclosed in US Patent Application Publication Nos. 20040149580 and 20040144658 to Flory. In one embodiment of the present disclosure, one or more electrodes can be used to form a resonant tunneling electrode configured to obtain data from analytes such as polymers which travel through a microchannel of the microfluidic device. The term “resonant” or “resonant tunneling” refers to an effect where the relative energy levels between the current carriers in the electrodes are relatively similar to the energy levels of the proximal analyte or polymer segment. This provides for increased conductivity. Resonant tunneling electrodes measure or detect tunneling current, for example from one electrode through an analyte such as a biopolymer to another electrode.

The resonant tunneling electrode can be formed in whole or part of one or more of a variety of electrically conductive materials including but not limited to, electrically conductive metals and alloys. Exemplary metals and alloys include, but are not limited to, tin, copper, zinc, iron, magnesium, cobalt, nickel, silver, platinum, gold, and/or vanadium. Other materials well known in the art that provide for electrical conduction may also be employed. In operation, an analyte such as a biopolymer is generally positioned sufficiently close to the resonant tunneling electrode so specific monomers and their sequence in the biopolymer can be detected and identified. It will be appreciated that the resonant tunneling electrode can be fitted to the shape and configuration of a curve or bend in a non-linear portion of a microchannel. Accordingly, the resonant tunneling electrode can be curved parts of rings or other shapes may be used with on in a microchannel. The electrodes may also be designed in broken format or spaced from each other. However, the electrodes should be capable of detecting a potential, conductance, or tunneling current as analytes pass through the space separating the electrodes.

Exemplary Methods of Use

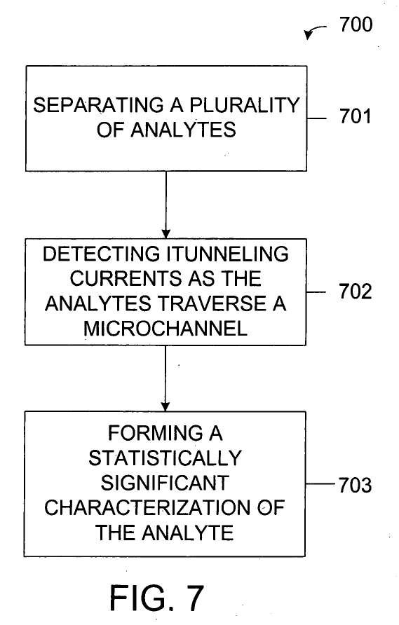

FIG. 7 shows an exemplary method 700 for characterizing an analyte. The process 700 begins by separating a mixture of analytes in step 701. In step 702, tunneling current from each analyte is detected as the analytes travel through a microchannel. Once date is collected, statistically significant characterization of the analyte can be formed according to step 703, based on the detected tunneling conductivity. In one embodiment, the characterization of the analyte comprises determining the sequence of a polymer, for example a biopolymer.

In general, sequencing involves detecting monomers of a polymer as the polymer moves down a voltage gradient established between two regions separated by the detection device 145. The detection device 145 is capable of interacting sequentially with the individual monomer residues of a polynucleotide present in one of the regions. Measurements are continued over time, as individual monomer residues of the polynucleotide interact sequentially, yielding data suitable to infer a monomer-dependent characteristic of the polynucleotide. In some embodiments, the monomer-dependent characterization achieved with the disclosed microfluidic system 100 may include identifying physical characteristics such as, but not limited to, the number and composition of monomers that make up each individual polynucleotide, in sequential order.

The term “sequencing” as used herein means determining the sequential order of monomers in a polymer, for example nucleotides in a polynucleotide molecule. Sequencing as used herein includes in the scope of its definition, determining the nucleotide sequence of a polynucleotide in a de novo manner in which the sequence was previously unknown. Sequencing as used herein also includes in the scope of its definition, determining the nucleotide sequence of a polynucleotide wherein the sequence was previously known. Sequencing polynucleotides, the sequences of which were previously known, may be used to identify a polynucleotide, to confirm a polynucleotide, or to search for polymorphisms and genetic mutations.

Biopolymers sequenced by the microfluidic system 100 can include polynucleotides comprising a plurality of nucleotide monomers, for example nucleotide triphosphates (NTPs). The nucleotide triphosphates can include naturally occurring and synthetic nucleotide triphosphates. The nucleotide triphosphates can include, but are not limited to, ATP, DATP, CTP, dCTP, GTP, dGTP, UTP, TTP, dTTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, 2-thiocytidine as well as the alphathiotriphosphates for all of the above, and 2′-O-methyl-ribonucleotide triphosphates for all the above bases. Preferably, the nucleotide triphosphates are selected from the group including dATP, dCTP, dGTP, dTTP, dUTP, and combinations thereof. Modified bases can also be used instead of or in addition to nucleotide triphosphates and can include, but are not limited to, 5-Br-UTP, 5-Br-dUTP, 5-F-UTP, 5-F-dUTP, 5-propynyl dCTP, and 5-propynyl-dUTP. Additionally, the nucleotides can be labeled with a detectable label, for example a label that modulates resonant tunneling current including, but not limited to, metal particles of about 100 nm in diameter or less.

One embodiment provides methods of sequencing a nucleic acid. In the methods, the biochemical components of a sequencing reaction including, but not limited to, a target nucleic acid, a first and optionally, second sequencing primer, a polymerase (optionally including thermostable polymerases for use in PCR, dNTPs, and ddNTPs) are mixed in a microfluidic device under conditions permitting target dependent polymerization of the dNTPs. Polymerization products are separated in the microfluidic device to provide a sequence of the target nucleic acid. Typically, sequencing information acquired by this method is used to select additional sequencing primers and/or templates, and the process is reiterated. Generally, a second sequencing primer is selected based upon the sequence of the target nucleic acid and the second sequencing primer is mixed with the target nucleic acid in a microfluidic device under conditions permitting target dependent elongation of the selected second sequencing primer, thereby providing polymerization products which are separated by size in the microfluidic device to provide further sequence of the target nucleic acid. The nucleic acids are electrophoretically stacked into bands 414 where the polymers in a single band are of uniform charge-to-mass ratio or size and have the same sequence of monomers. Each band then is analyzed by the detection device 145.

It should be emphasized that many variations and modifications may be made to the above-described embodiments. All such modifications and variations are intended to be included herein within the scope of this disclosure and protected by the following claims.

What is claimed is:

1. A microfluidic system comprising:
a sample preparation device; and
a microfluidic in fluid communication with the sample preparation device, the microfluidic device comprising at least one set of resonant tunneling electrodes disposed in a microchannel, the resonant tunneling electrodes configured to detect an analyte.

2. The microfluidic system of claim 1, further comprising:
a material transport system configured to transport the analyte through the microchannel.

3. The microfluidic system of claim 2, wherein the material transport system comprises at least one of the following: electrokinetic components, electroosmotic components, electrophoretic movement components, micro-pumps, microvalves, fluid switches, fluid gates, and combinations thereof.

4. The microfluidic system of claim 1, further comprising:
a power source coupled to the resonant tunneling electrodes and configured to supply an electric field to an analyte in the microfluidic system.

5. The microfluidic system of claim 1, further comprising:
a computer system configured to control operation of the microfluidic system and storing acquired data, wherein the computer system is communicatively coupled to at least one of the following: the sample preparation device, the microfluidic device, the resonant tunneling electrodes, and combinations thereof.

6. The microfluidic system of claim 1, wherein the microchannel comprises a separation matrix for sorting the analyte into components within the analyte.

7. The microfluidic system of claim 1, wherein the sample preparation device comprises an electrophoretic device.

8. The microfluidic system of claim 1, wherein the set of resonant tunneling electrodes comprises two electrodes separated by a distance of about 2 nm or more.

9. The microfluidic system of claim 1, wherein the microchannel is less than 1 mm in at least one dimension.

10. The microfluidic system of claim 1, wherein the microchannel is about 1.0 to about 150 μm in at least one dimension.

11. The microfluidic system of claim 1, wherein the analyte is selected from: DNA, RNA, polypeptides, polynucleotides, and combinations thereof.

12. A method for sequencing a target polynucleotide, the method comprising:
electrophoretically separating a plurality of polynucleotides based on at least one characteristic of the polynucleotides;
receiving the separated polynucleotides into a microfluidic channel; and
determining a statistically significant sequence of the target polynucleotide by detecting tunneling current through the each of the polynucleotides with a resonant tunneling electrode and correlating the detected current to predetermined currents indicative of specific polynucleotides.

13. A microfluidic system comprising:
a capillary electrophoretic device operably coupled to a microchannel of a microfluidic device, the microchannel configured to received electrophoretically separated analytes; and
a resonant tunneling electrode disposed in the microchannel for detecting at least one of the separated analytes.

14. The microfluidic system of claim 13, further comprising:
a material transport system operatively coupled to the microchannel configured to transport the analytes through the microchannel.

15. The microfluidic system of claim 13, wherein the material transport system comprises at least one of the following: electrokinetic components, electroosmotic components, electrophoretic movement components, micro-pumps, microvalves, fluid switches, fluid gates, and combinations thereof.

16. The microfluidic system of claim 13, further comprising:
a power source operatively coupled to the resonant tunneling electrodes and configured to supply an electric field to a sample in the microfluidic system.

17. The microfluidic system of claim 13, further comprising:
a computer system configured to control operation of the microfluidic system and storing acquired data, wherein the computer system operatively coupled to at least one of the following: the electrophoretic device, the microchannel, the resonant tunneling electrodes, and combinations thereof.

18. The microfluidic system of claim 13, wherein the microchannel comprises a separation matrix configured to sort a sample.

19. The microfluidic system of claim 13, wherein the resonant tunneling electrode comprises two electrodes separated by a distance of about 2 nm.

20. The microfluidic system of claim 13, wherein the microchannel is less than 1 mm in at least one dimension.

21. The microfluidic system of claim 13, wherein the microchannel is about 1.0 to about 150 μm in at least one dimension.
